(19) United States
(12) Patent Application Publication
(10) Pub. No.: US 2011/0213256 A1
(43) Pub. Date: Sep. 1, 2011

(54) BLOOD PRESSURE MEASUREMENT DEVICE

(75) Inventors: Minoru Taniguchi, Kuze-gun (JP); Yoichiro Watanabe, Otsu-shi (JP); Lanlan Wang, Dailian (CN); Yuuichi Noro, Matsusaka-shi (JP)

(73) Assignee: OMRON HEALTHCARE CO., LTD., Kyoto-shi (JP)

(21) Appl. No.: 13/104,668

(22) Filed: May 10, 2011

Related U.S. Application Data
(63) Continuation of application No. PCT/JP2009/068457, filed on Oct. 28, 2009.

(30) Foreign Application Priority Data
Nov. 10, 2008 (JP) 2008-287842

(51) Int. Cl. A61B 5/0225

(52) U.S. Cl. 600/493

(57) ABSTRACT

In a blood pressure measurement device, a force acts on one end side and the other end side of a cuff main body in a direction where an inner diameter of a tubular form is further reduced, so that a rotation shaft rotates in a clockwise direction thus simultaneously moving the one end side and the other end side of the cuff main body. As a result, the inner diameter of the tubular form of the cuff main body can be reduced, and the cuff main body can be easily and rapidly wrapped around the upper arm with only the pushing task using one hand. Thus, the blood pressure measurement device provides a simple device configuration that enables the cuff to be easily wrapped around the measurement target site even for those with crippled hands.

### TECHNICAL FIELD

The present invention relates to a blood pressure measurement device for measuring blood pressure by wrapping a cuff band around the arm.

### BACKGROUND ART

When measuring the blood pressure, an air bag is wrapped around to compress the artery of the measurement target site (upper arm, wrist, femoral area, ankle) in the human body, and then the air bag is restrained from the periphery to be fixed, and then pressurized or depressurized to measure the blood pressure.

Japanese Unexamined Patent Publication No. 2005-230175 (patent document 1) discloses a blood pressure measurement device including an automatic cuff winding mechanism. Japanese Unexamined Patent Publication No. 2008-054867 (patent document 2) discloses a blood pressure measurement device performed by wrapping the cuff band around the upper arm using the force of the hand. In either blood pressure measurement device, the blood pressure is measured by sending air to the air bag arranged inside the cuff band, and compressing the upper arm.

In the blood pressure measurement device disclosed in patent document 1, however, the mechanism for automatically wrapping the cuff up to the wrapping size (wrapping peripheral length) of the measurement target site is complicated and may increase the cost of the blood pressure measurement device.

In the case of the wrapping configuration using the force of the hand described in patent document 2, delicate operation of the hand is required for the adjustment operation of the wrapping strength of the cuff, and the like, and hence, such device is inconvenient for those with crippled hands.

Patent Document 1: Japanese Unexamined Patent Publication No. 2005-230175

Patent Document 2: Japanese Unexamined Patent Publication No. 2008-054867

### SUMMARY OF INVENTION

Therefore, one or more embodiments of the present invention provides a blood pressure measurement device with a simple device configuration and having a configuration enabling the cuff to be easily wrapped around the site to be measured even for those with crippled hands.

A blood pressure measurement device according to one or more embodiments of the present invention is a blood pressure measurement device including a cuff with an air bag for compressing an artery of a measurement target site and used by being attached to a site to be measured at the time of measurement, wherein the cuff includes a cuff main body having a band-like form in a developed state, and being rounded to a tubular form so that one end side and the other end side overlap each other to receive the measurement target site from an axial direction; and an engagement rotation member arranged at a position sandwiched by the one end side and the other end side at a position where the one end side and the other end side of the cuff main body overlap each other with the cuff main body rounded, and arranged to be rotatable in a direction where an inner diameter of the tubular form reduces or in a direction where the inner diameter is enlarged while engaging with the one end side and the other end side of the cuff main body; and the cuff main body includes a flexible member for maintaining the tubular form with the cuff main body being rounded.

In a blood pressure measurement device according to one or more embodiments of the present invention, the one end side and the other end side of the cuff main body can be simultaneously moved in a direction where the inner diameter of the tubular form is reduced or in a direction where the inner diameter is enlarged according to the rotation of the engagement rotation member, and the cuff main body can be easily and rapidly wrapped around the site to be measured by arranging the engagement rotation member that engages with the one end side and the other end side of the cuff main body with the cuff main body being rounded.

If an external force against the elastic force to enlarge the diameter of the rounded flexible member is released, the inner diameter of the tubular form of the cuff main body can be rapidly enlarged based on the elastic force of the flexible member.

For instance, if an external force towards the engagement rotation member side is acted on the outer peripheral surface on the upper side of the cuff main body at the position on the opposite side of the engagement rotation member against the elastic force to enlarge the diameter of the rounded flexible member, the one end side and the other end side of the cuff main body can be simultaneously moved in the direction where the inner diameter of the tubular form is further reduced. As a result, the cuff main body can be easily and rapidly wrapped around the site to be measured.

In a blood pressure measurement device according to one or more embodiments of the present invention, a blood pressure measurement device with a simple device configuration and having a configuration enabling the cuff main body to be easily wrapped around the measurement target site even for those with crippled hands is provided.

### BRIEF DESCRIPTION OF THE DRAWINGS

FIG. 1 is a view showing an outer appearance structure of a blood pressure measurement device according to a first embodiment.

FIG. 2 is a view showing an outer appearance structure showing a state in which an upper arm is arranged in the blood pressure measurement device according to the first embodiment.

FIG. 3 is a cross-sectional view corresponding to the view taken along line III-Ill in FIG. 1.

FIG. 4 is a developed view showing the structure of the cuff main body in the first embodiment.

FIG. 5 is a cross-sectional view corresponding to the view taken along line V-V in FIG. 4.

FIG. 6 is a view showing an inner structure of the case body in the first embodiment.

FIG. 7 is a first view showing the operation of a lock/unlock mechanism accommodated inside the case body in the first embodiment.

FIG. 8 is a second view showing the operation of the lock/unlock mechanism accommodated inside the case body in the first embodiment.

FIG. 9 is a cross-sectional view showing a diameter reduced state corresponding to the view taken along line in FIG. 1.

FIG. 10 is a view showing an internal structure of the blood pressure measurement device according to a second embodiment.

FIG. 11 is a schematic view showing a control block diagram of the blood pressure measurement device according to the second embodiment.

FIG. 12 is a view showing a flow of the blood pressure measurement using the blood pressure measurement device according to the second embodiment.

FIG. 13 is a first view showing the relationship of stroke and pressure at the time of blood pressure measurement in the second embodiment.

FIG. 14 is a first schematic cross-sectional view showing a tightened state of the upper arm at the blood pressure measurement device according to the second embodiment at the time of blood pressure measurement in the second embodiment.

FIG. 15 is a second view showing the relationship of stroke and pressure at the time of blood pressure measurement in the second embodiment.

FIG. 16 is a second schematic cross-sectional view showing a tightened state of the upper arm at the blood pressure measurement device according to the second embodiment.

FIG. 17 is a view showing an outer structure of the blood pressure measurement device according to a third embodiment.

FIG. 18 is a view showing a structure of a cuff of the blood pressure measurement device according to the third embodiment.

FIG. 19 is a cross-sectional view corresponding to a view taken along line XIX-XIX in FIG. 18.

FIG. 20 is a schematic plan view showing a rotation mechanism including an engagement rotation member of the blood pressure measurement device in the third embodiment.

FIG. 21 is a block diagram of the blood pressure measurement device according to the third embodiment.

### DETAILED DESCRIPTION OF INVENTION

Each embodiment of the blood pressure measurement device according to the present invention will be described in detail below with reference to the drawings. The blood pressure measurement device according to the embodiment shown below is a so-called upper arm type blood pressure measurement device in which the upper arm is adopted for the site to be measured. However, the essence of the present invention is not limited to the upper arm type blood pressure measurement device, and can be applied to a blood pressure measurement device aimed to measure the blood pressure by wrapping the air bag to compress the artery of the site to be measured (upper arm, wrist, femoral area, ankle) of the human body, restraining the air bag from the periphery to fix it, and pressurizing or depressurizing the air bag.

### First Embodiment

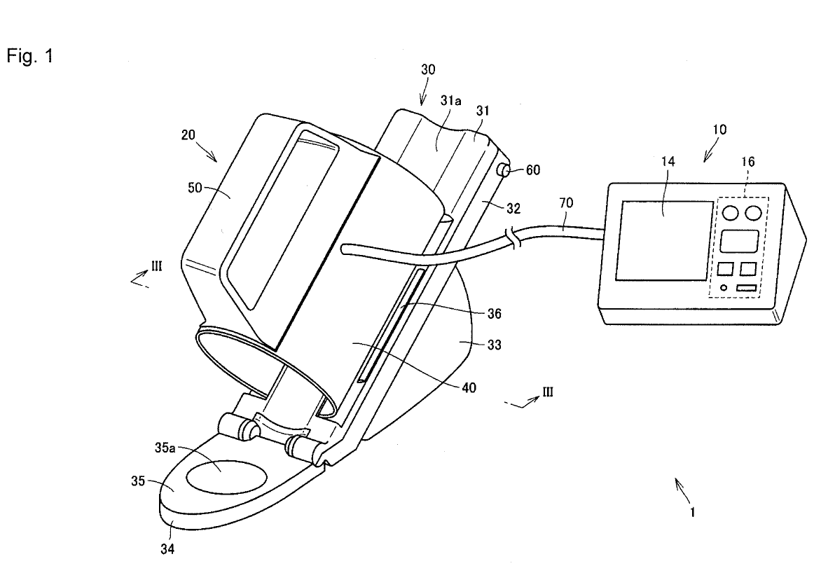

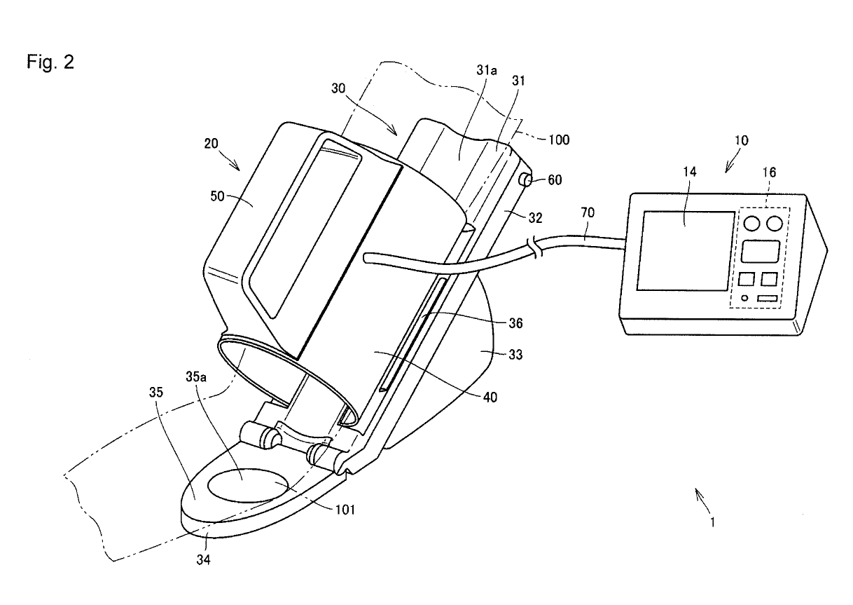

An outer appearance structure of a blood pressure measurement device 1 according to a first embodiment will be described with reference to FIG. 1 and FIG. 2. FIG. 1 is a view showing the outer appearance structure of the blood pressure measurement device 1 according to the present embodiment. FIG. 2 is a view showing the outer appearance structure showing a state in which the upper arm is arranged in the blood pressure measurement device 1 according to the present embodiment.

As shown in FIG. 1 and FIG. 2, the blood pressure measurement device 1 according to the present embodiment includes a main body 10, a cuff 20 and an air tube 70. The main body 10 is used by being placed on a mounting surface of a table and the like at the time of the measurement, and includes a display unit 14 and an operation unit 16 on the upper surface. The cuff 20 is used by being attached to the upper arm while being placed on the mounting surface of the table and the like at the time of the measurement, and includes an upper arm supporting stand 30 and a cuff main body 40.

The air tube 70 is a member for coupling the main body 10 and the cuff 20, which are separately configured, and is configured by a flexible tube. The configuration in which the main body 10 and the cuff 20 are separated is not the sole case, and embodiments of the present invention can also be applied to a blood pressure measurement device in which the main body 10 and the cuff 20 are integrated.

The upper arm supporting stand 30 of the cuff 20 includes an upper arm supporting surface 31 with a curved surface 31a to which the upper arm 100 is arranged at the time of measurement, and a case body 32, a seat 33 arranged on the lower side of the case body 32, and an elbow placement section 34 arranged at the front side of the lower end of the case body 32.

The upper arm supporting stand 30 is arranged so that the case body 32 is tilted at the upper part of the seat 33 in such manner that the upper arm supporting surface 31 is tilted when mounted on the mounting surface. The elbow placement section 34 includes an elbow placement surface 35, where a switch 35a for detecting that the elbow is placed is arranged at the central part.

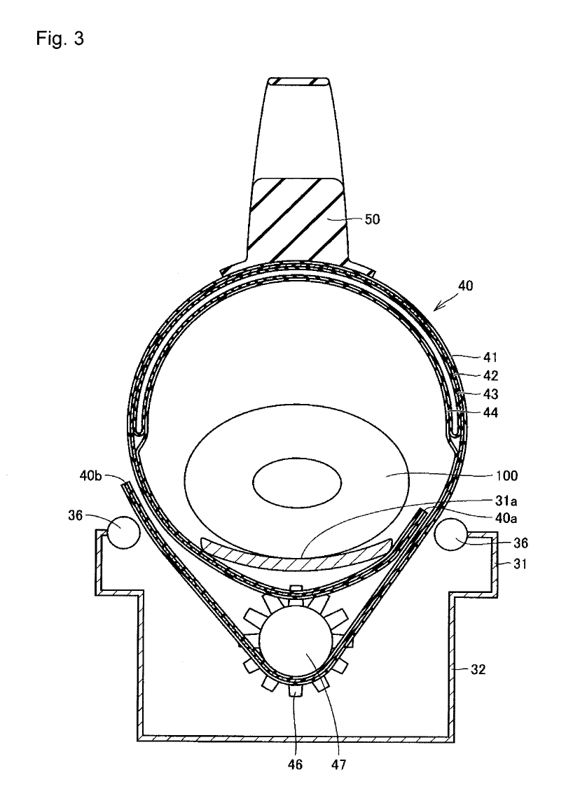

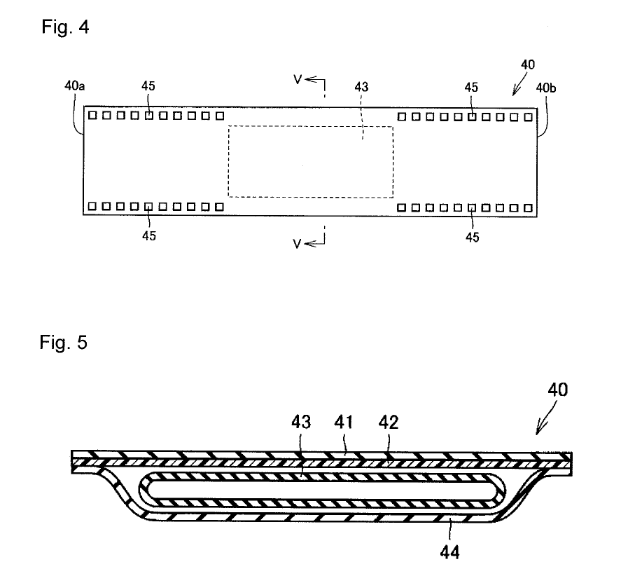

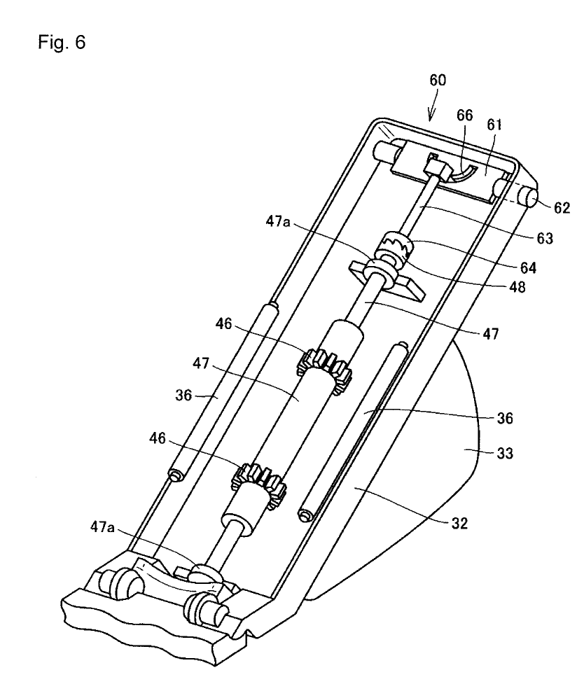

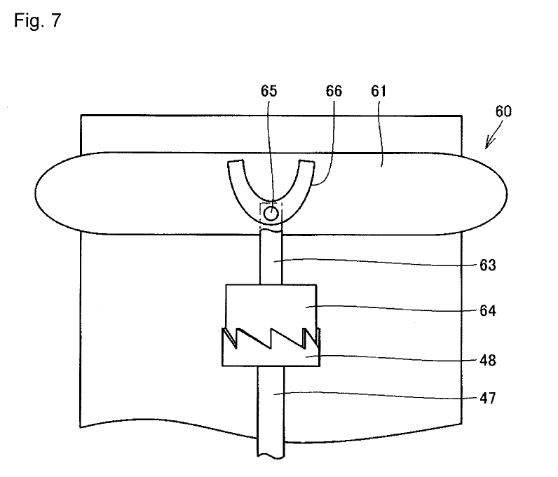

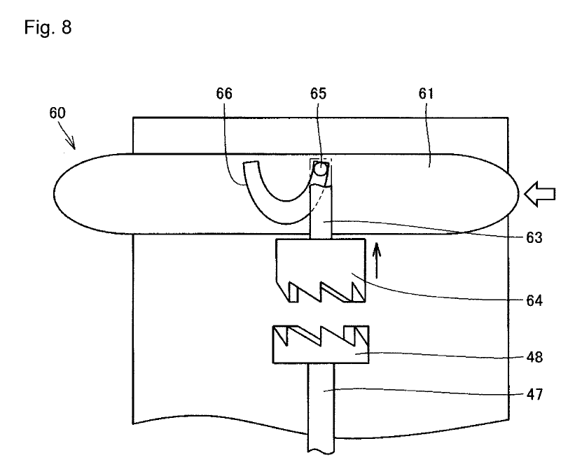

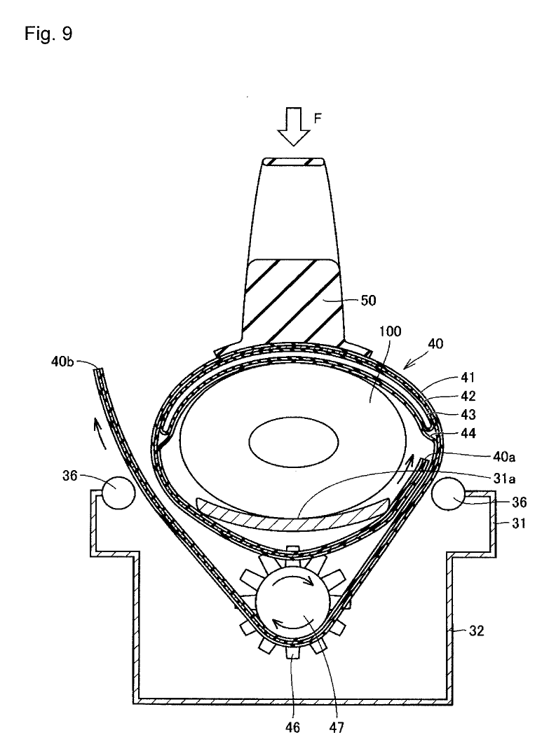

A specific configuration of the cuff 20 will now be described with reference to FIG. 3 to FIG. 9. FIG. 3 is a cross-sectional view corresponding to the view taken along line III-III in FIG. 1, FIG. 4 is a developed view showing the structure of the cuff main body, FIG. 5 is a cross-sectional view corresponding to the view taken along line V-V in FIG. 4, FIG. 6 is a view showing an inner structure of the case body 32, FIG. 7 and FIG. 8 are first and second views showing the operation of a lock/unlock mechanism accommodated inside the case body 32, and FIG. 9 is a cross-sectional view showing a diameter reduced state corresponding to the view taken along line III-III in FIG. 1.

As shown in FIG. 3 and FIG. 4, the cuff 20 includes the cuff main body 40, and a gearing tooth 46 serving as an engagement rotation member. The cuff main body 40 has a band-like form in the developed state, and is rounded to a tubular form so that one end side 40a and the other end side 40b overlap to enable the upper arm 100, which is the site to be measured, to be inserted from the axial direction. A handle 50 is arranged on an outer peripheral surface on the upper side of the cuff main body 40 on the side opposite to the gearing tooth 46.

As shown in FIG. 5, the cuff main body 40 includes a flexible member 42 for maintaining the tubular form as a core member with the cuff main body 40 being rounded, the flexible member 42 being covered by an outer cloth 41 and an inner cloth 44. The flexible member 42 may use PP (polypropylene), PS (polystyrene), PET (polyethylene terephthalate), SUS (stainless steel), and the like having a thickness of about 2 mm.

An air bag 43 for compressing the artery of the upper arm 100 is accommodated at a position sandwiched by the flexible member 42 and the inner cloth 44 at a central portion of the cuff main body 40. The air tube 70 is connected to the air bag 43.

A plurality of engagement holes 45, to which the gearing tooth 46 (to be described later) gears with, is arranged along the longitudinal direction of the cuff main body 40 at one end side 40a and the other end side 40b of the cuff main body 40. In the present embodiment, the plurality of engagement holes 45 is arranged along two sides in the longitudinal direction of the cuff main body 40.

With reference again to FIG. 3, the gearing tooth 46 serving as the engagement rotation member is arranged at the position sandwiched by the one end side 40a and the other end side 40b at the position where the one end side 40a and the other end side 40b of the cuff main body 40 overlap each other with the cuff main body 40 being rounded to a cylindrical shape. The gearing tooth 46 is positioned inside the case body 32 at the lower side of the curved surface 31a.

As shown in FIG. 6, more specifically, a rotation shaft 47 is arranged to extend in the up and down direction along the inclined surface. The upper end and the lower end of the rotation shaft 47 are respectively shaft supported so as to be rotatable about a shaft by a bearing member 47a. The plurality of gearing teeth 46 are arranged at two areas in the axial direction of the rotation shaft 47 so as to gear with the plurality of engagement holes 45 arranged along two sides in the longitudinal direction of the cuff main body 40.

A guide roller 36 for supporting the cuff main body 40 from both sides is arranged at both sides of the case body 32 so as to sandwich the gearing tooth 46 from the side surface side.

In the present embodiment, a case where the gearing tooth 46 is arranged at twelve areas about the rotation shaft 47 is described, but the number is merely illustrative and the numerical quantity may be appropriately changed. The gearing tooth 46 is arranged at two areas with a predetermined spacing in the axial direction of the rotation shaft 47, but this is to send the cuff main body 40 in a parallel state, and thus, it is not necessarily limited to two areas and a configuration of arranging only at one area by arranging a member etc. for guiding the transporting direction of the cuff main body 40 may be adopted, or a configuration of arranging at three or more areas to more stably send the cuff main body 40 may be adopted.

As shown in FIG. 7, the upper end portion of the rotation shaft 47 includes a lock/unlock mechanism 60 capable of selecting between a state (state in which the lock state is released to be described later) of allowing the rotation in the direction where the inner diameter of the tubular shape of the cuff main body 40 is reduced, and a state of inhibiting the rotation in the direction where the inner diameter of the tubular shape is enlarged. The lock/unlock mechanism 60 includes a slide button 61 that slidably moves in the horizontal direction, where a U-shaped groove 66 is arranged at the central region of the slide button 61. An engagement pin 65 that engages with the groove 66 is inserted into the groove 66, and the engagement pin 65 is attached to an engagement shaft 63 extending in the up and down direction. Clutch mechanisms 48, 64 are attached between the engagement shaft 63 and the rotation shaft 47.

As shown in FIG. 8, when the slide button 61 of the lock/unlock mechanism 60 is slid in the horizontal direction, the engagement pin 65 moves along the groove 66, and the engagement shaft 63 slides to the upper side of the shaft direction, by which the clutch mechanisms 48, 64 can be separated.

With reference again to FIG. 3, the cuff main body 40 is in a state of being rounded to the tubular form, where the state shown in FIG. 3 is the initial state. When reducing the inner diameter of the tubular form, the force acts in the direction where the inner diameter of the tubular form is enlarged based on the elastic force of the flexible member 42. In this case, the force of rotating in the counterclockwise direction acts on the rotation shaft 47 shown in FIG. 3 through the gearing tooth 46, but the rotation of the rotation shaft 47 is inhibited by the lock/unlock mechanism 60, and the tubular form of the cuff main body 40 is maintained.

In the blood pressure measurement device 1 having the above configuration, the state shown in FIG. 3 is the state in which the inner diameter of the cylindrical shape of the cuff main body 40 is most enlarged, and the upper arm 100 is inserted from the axis direction of the cylindrical shape of the cuff main body 40 from such state until placing the elbow at the elbow placement section 34, as shown in FIG. 2.

Thereafter, as shown in FIG. 9, the handle 50 is gripped with the hand on the opposite side of the inserted upper arm to act as an external force (direction indicated with F in FIG. 9) directed towards the gearing tooth 46 side against the elastic force of enlarging the diameter of the rounded flexible member 42. If the handle 50 is not so arranged, the external force towards the direction indicated with F in FIG. 9 is directly acted using the palm on the opposite side of the inserted upper arm.

The force in the direction of further reducing the inner diameter of the tubular form thus acts on the one end side 40a and the other end side 40b of the cuff main body 40, so that the rotation shaft 47 rotates in the clockwise direction and the one end side 40a and the other end side 40b of the cuff main body 40 are simultaneously moved. As a result, the inner diameter of the tubular form of the cuff main body 40 can be reduced, and the cuff main body 40 can be easily and rapidly wrapped around the upper arm with only the pushing task using one hand. Furthermore, the wrapped state of the cuff main body 40 can be maintained by acting the lock/unlock mechanism 60.

Because the position of the air bag 43 arranged in the cuff main body 40 does not change when wrapping the cuff main body 40 around the upper arm 100, the artery of the upper arm 100 can be accurately compressed, and the accuracy of the blood pressure measurement can be enhanced.

At the end of the blood pressure measurement, the slide button 61 of the lock/unlock mechanism 60 is slid in the horizontal direction so that the engagement pin 65 moves along the groove 66, and the engagement shaft 63 slides to the upper side in the axis direction thereby separating the clutch mechanisms 48, 64, as shown in FIG. 8. As a result, the cuff main body 40 can easily restore the initial state shown in FIG. 3 when the inner diameter of the tubular form is rapidly enlarged based on the elastic force of enlarging the diameter of the rounded flexible member 42.

### Second Embodiment

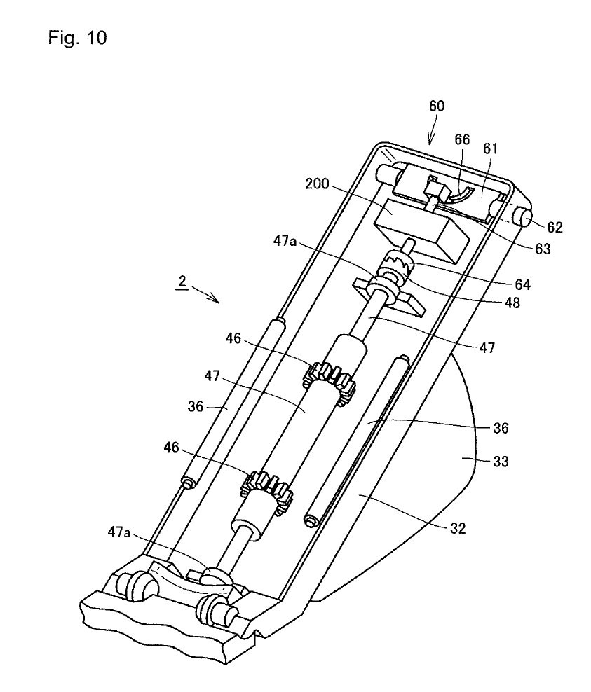

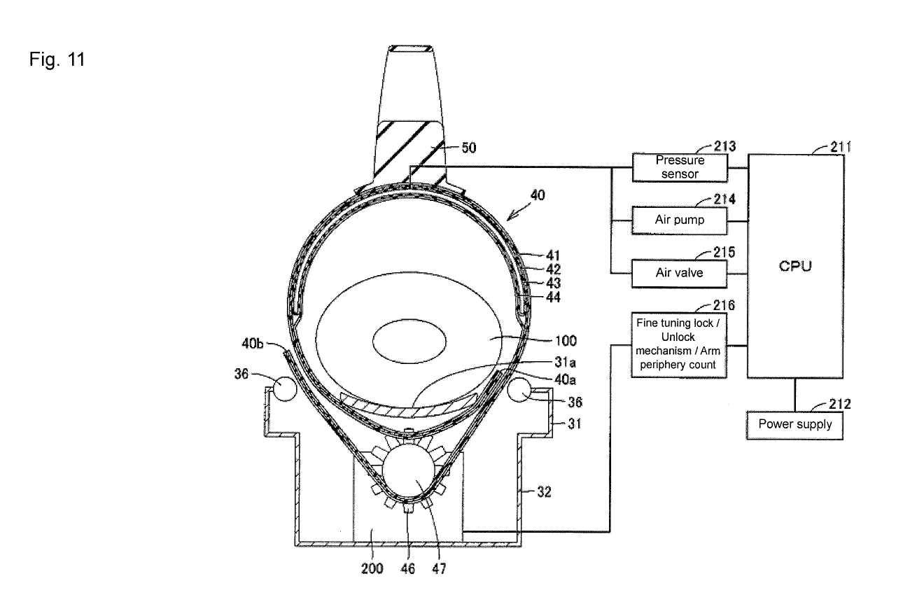

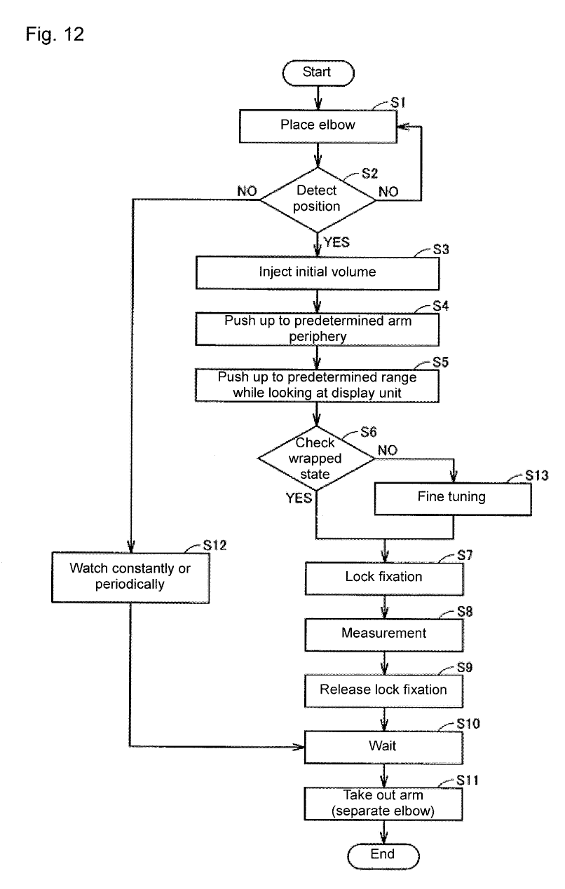

A blood pressure measurement device 2 according to a second embodiment of the present invention will now be described based on FIG. 10 to FIG. 16. FIG. 10 is a view showing an internal structure of the blood pressure measurement device 2 according to the present embodiment, FIG. 11 is a schematic view showing a control block diagram of the blood pressure measurement device 2 according to the present embodiment, and FIG. 12 is a view showing a flow of the blood pressure measurement using the blood pressure measurement device 2 in the second embodiment of the present invention.

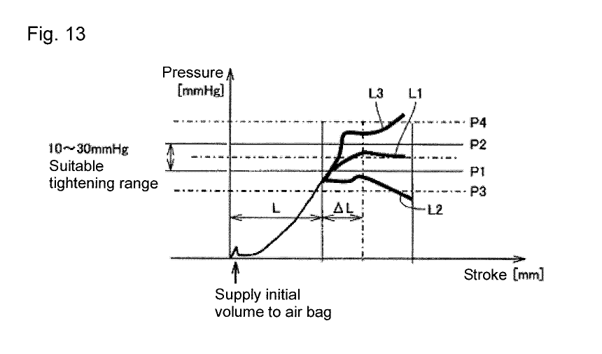

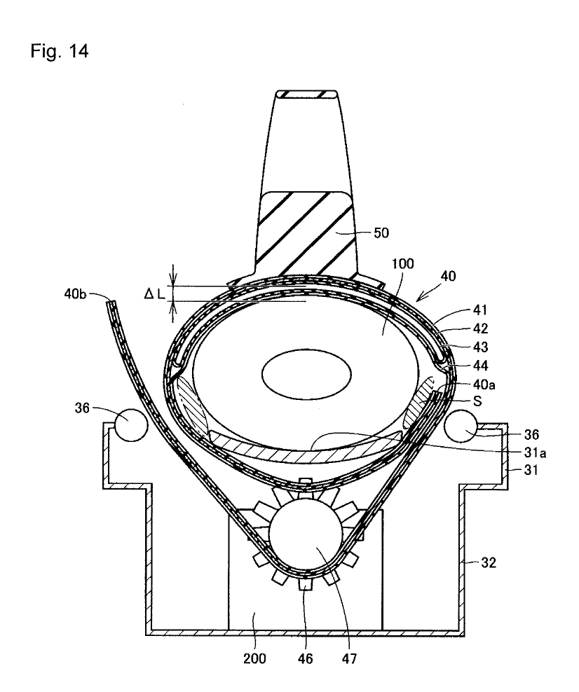

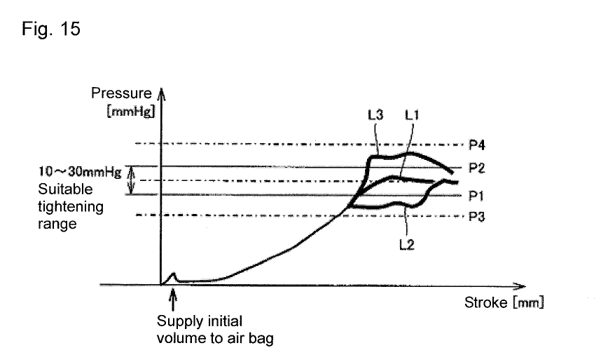

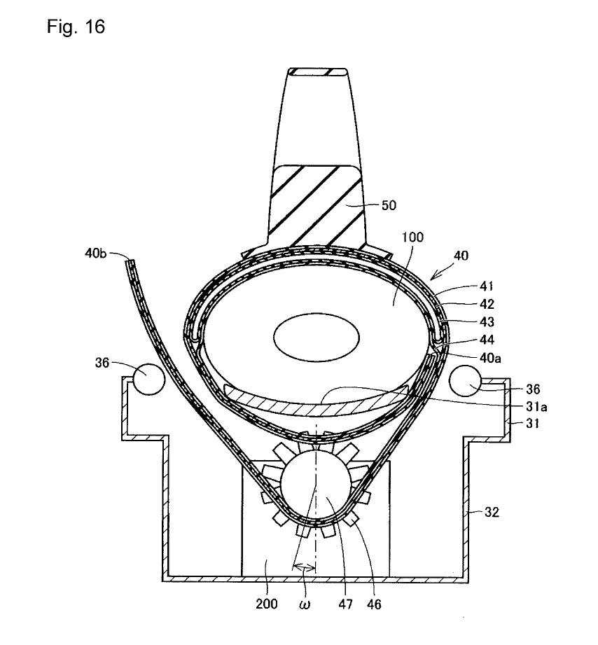

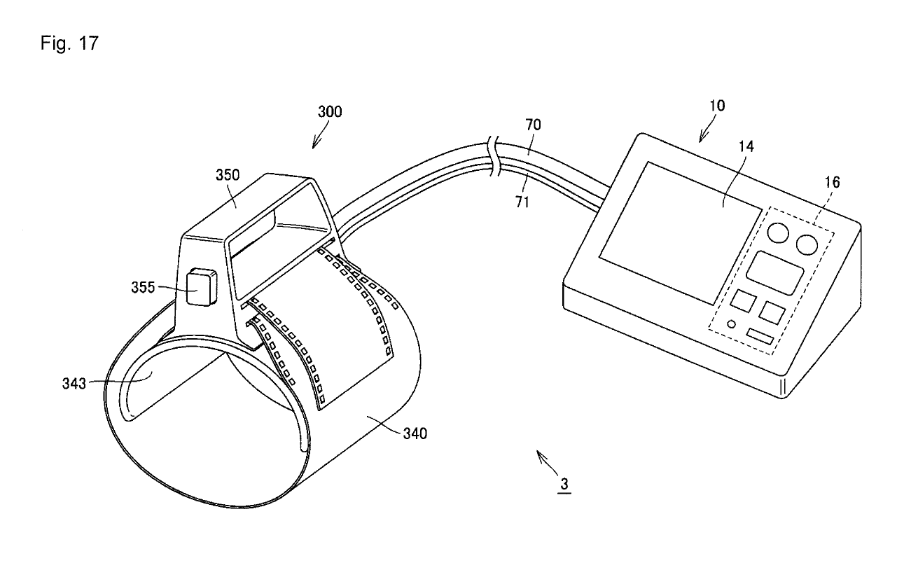

FIG. 13 is a first view showing the relationship of stroke and pressure at the time of blood pressure measurement, FIG. 14 is a first schematic cross-sectional view showing a tightened state of the upper arm 100 at the blood pressure measurement device 2 according to the second embodiment at the time of blood pressure measurement, FIG. 15 is a second view showing the relationship of stroke and pressure at the time of blood pressure measurement, and FIG. 16 is a second schematic cross-sectional view showing a tightened state of the upper arm 100 at the blood pressure measurement device 2.

A case in which the cuff main body 40 is manually wrapped around the upper arm 100 in the blood pressure measurement device 1 according to the first embodiment has been described, but in the blood pressure measurement device 2 according to the second embodiment, a torque motor 200 is coupled as one example of a drive device to the rotation shaft 47 to wrap the cuff main body 40 around the upper arm 100 using the force of the motor, as shown in FIG. 10.

Other configurations are the same as the configurations in the first embodiment, and thus, the same reference numbers are denoted for the same or corresponding portions, and the redundant description will not be repeated. The lock/unlock mechanism 60 may not necessarily be used because the rotating direction of the rotation shaft 47 can be controlled using the torque motor 200. However, according to one or more embodiments of the present invention, the lock/unlock mechanism 60 is arranged when adopting a configuration of forcibly separating the torque motor 200 and the rotation shaft 47.

As shown in FIG. 11, a pressure sensor 213, an air pump 214, and an air valve 215 are coupled to the air bag 43. The CPU 211 controls the pressure sensor 213, the air pump 214, and the air valve 215. A fine tuning lock/unlock mechanism/arm periphery count 216 is coupled to the torque motor 200 with lock function, and the fine tuning lock/unlock mechanism/arm periphery count 216 is controlled by the CPU (Central Processing Unit) 211. A power supply unit 212 is coupled to the CPU 211.

The flow of the blood pressure measurement using the blood pressure measurement device 2 according to the second embodiment of the present invention will now be described with reference to FIG. 12. First, the upper arm 100 is inserted to the cuff main body 40, and the elbow is placed at the elbow placement section 34 (step 1). The placement of the elbow is checked by the switch 35a (step 2). The process returns to step 1 if the placement of the elbow is not confirmed.

After the placement of the elbow is confirmed, the air of an initial volume is introduced to the air bag 43 (step 3). Thereafter, the handle 50 is gripped with the hand on the opposite side of the inserted upper arm and pushed to the position of a predetermined arm periphery, by which the inner diameter of the tubular form of the cuff main body 40 is reduced (step 4). Furthermore, the inner diameter of the tubular form of the cuff main body 40 is reduced until reaching the arm periphery of a predetermined range while checking the display unit 14 of the main body 10 (step 5). In step 4 and step 5, the inner pressure of the air bag 43 is checked with the pressurization sensor 213 to check the most suitable wrapped state (step 6). Fine tuning is performed if the wrapped state needs to be adjusted (step 13).

After checking the most suitable wrapped state, the lock of the torque motor 200 is fixed (step 7). The air is then introduced into the air bag 43 to perform the blood pressure measurement (step 8). The details of the blood pressure measurement will be described later. Thereafter, the lock fixation of the torque motor 200 is released (step 8) to transition to the waiting state (step 9). The placement of the elbow is detected constantly or periodically in step 2, and if the placement of the elbow is not detected, the state transitions to the waiting state.

After measuring the blood pressure, the elbow is moved away from the switch 35a, so that a check can be made that the upper arm 100 is taken out from the cuff main body 40 (step 11). The series of blood pressure measurement operation is thereby terminated.

The blood pressure measurement will now be described with reference to FIG. 13 to FIG. 16. First, with reference to FIG. 13 and FIG. 14, the amount of stroke (L) is measured with the torque motor 200 after the air of an initial volume is introduced to the air bag 43, and the rise in the inner pressure is determined with the slope of the inner pressure. The inner pressure of the air bag 43 is further monitored by very small stroke (ΔL).

In the graph of FIG. 13, the suitable tightening range is in the range of 10 mmHg (line P1) to 30 mmHg (line P2), where the tightening is loose if lower than the line P3 (0 to lower than or equal to 10 mmHg), and a space is formed between the cuff main body 40 and the upper arm 100 (space S shown in FIG. 14). The tightening is too tight if greater than the line P4 (30 to equal to 50 mmHg or less).

The tightened state shown with the line L1 is a suitable tightened state because the tightening pressure is positioned between the line P1 and the line P2. The tightened state shown with the line L2 is a wrapping abnormality because the tightening pressure is lower than the line P3. The tightened state shown with the line L3 is a wrapping abnormality since the tightening pressure is greater than the line P4. Therefore, the rotation amount (ω) of the torque motor 200 needs to be determined because a suitable tightened state cannot be obtained on a steady basis by simply providing a microscopic stroke (ΔL).

With reference to FIG. 15 and FIG. 16, a case where the rotation amount (ω) of the torque motor 200 is adjusted is shown. The rotation amount (ω) of the torque motor 200 is adjusted after providing the microscopic stroke (ΔL) so that the tightened pressure can be controlled within the range of the 10 mmHg (line P1) to 30 mmHg (line P2) in all cases shown with the lines L1, L2, L3.

### Third Embodiment

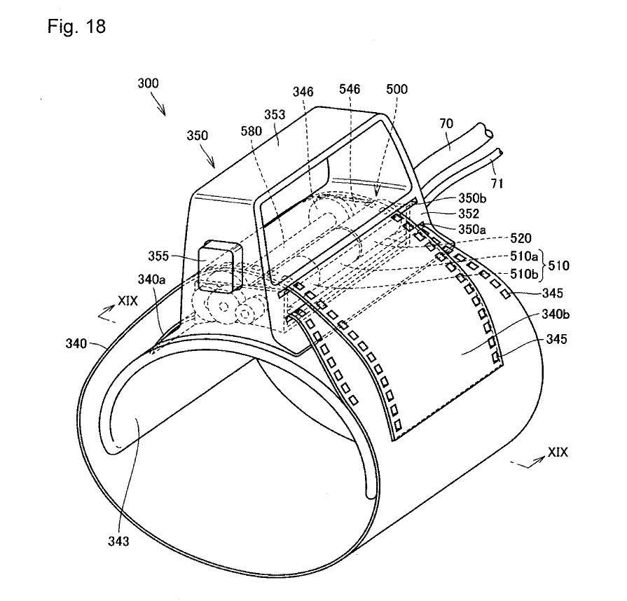

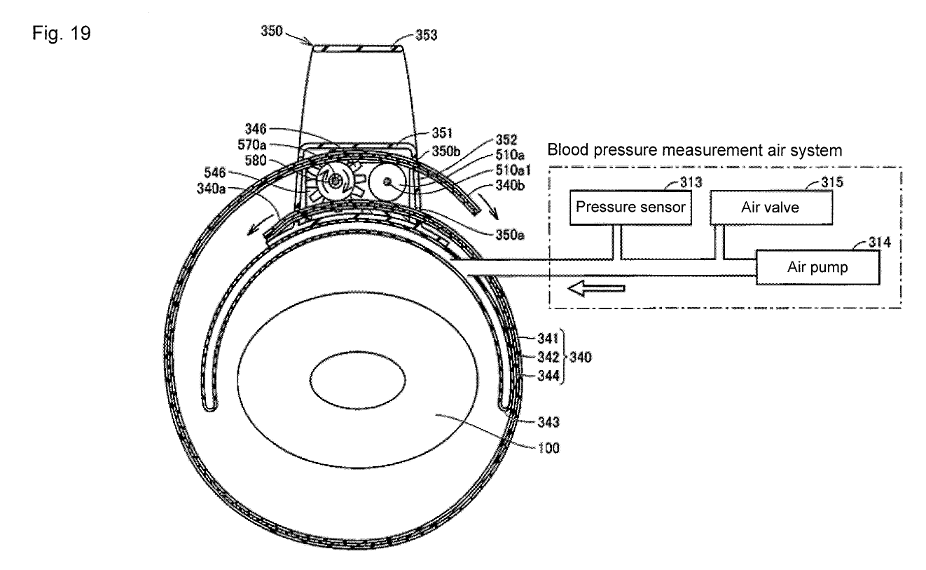

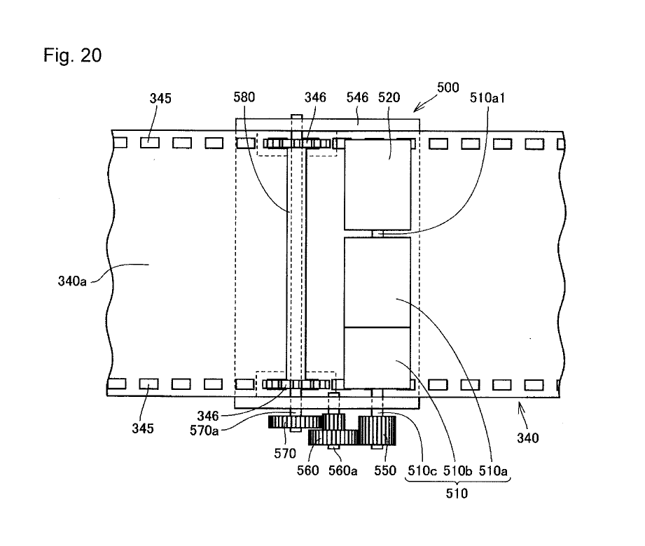

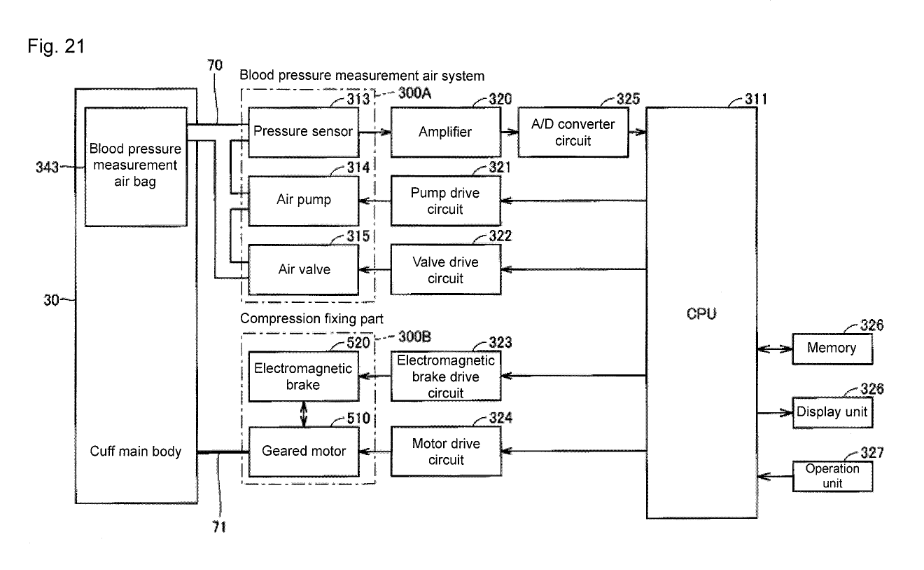

A blood pressure measurement device 3 according to a third embodiment of the present invention will now be described based on FIG. 17 to FIG. 21. FIG. 17 is a view showing an outer structure of the blood pressure measurement device 3 according to the present embodiment, and FIG. 18 is a view showing a structure of a cuff 300 of the blood pressure measurement device 3 according to the present embodiment. FIG. 19 is a cross-sectional view corresponding to a view taken along line XIX-XIX in FIG. 18, FIG. 20 is a schematic plan view showing a rotation mechanism including an engagement rotation member of the blood pressure measurement device 3 in the present embodiment, and FIG. 21 is a block diagram of the blood pressure measurement device 3 according to the present embodiment.

As shown in FIG. 17, the blood pressure measurement device 3 according to the present embodiment includes the main body 10, the cuff 300, the air tube 70, and a connection cable 71. The main body 10 has a box-shaped housing, and includes the display unit 14 and the operation unit 16 on the upper surface. The main body 10 is used by being placed on the mounting surface of a table and the like at the time of the measurement. The cuff 300 includes a tubular cuff main body 340 with a hollow opening to which the upper arm can be inserted from the axial direction, and a grip portion 350 arranged on the outer peripheral surface of the cuff main body 340. The cuff 300 is used by being attached on the upper arm 100 at the time of measurement. The air tube 70 and the connection cable 71 connect the main body 10 and the cuff 300, which are separately configured.

The detailed structure of the cuff 300 of the blood pressure measurement device according to the present embodiment will now be described with reference to FIG. 18 to FIG. 20.

As shown in FIG. 18 and FIG. 19, the cuff 300 includes the tubular cuff main body 340 to be attached to the upper arm 100, and the grip portion 350 arranged on the outer peripheral surface of the cuff main body 30. The grip portion 350 includes a case 351 for accommodating the rotation mechanism 500 including the gearing tooth 346 serving as the engagement rotation member, a base 352 for holding the air bag 343, and a grip 353 or a portion to be gripped upon attachment. A pressure sensor 313, an air pump 314, and an air valve 315 are coupled to the air bag 343.

The case 351 includes a first slit 350a that passes one end side 340a of the cuff main body 340 therethrough, and a second slit 350b, positioned on the upper side of the first slit 350a, that passes the other end side 340b of the cuff main body 340 therethrough. The push button 355 is arranged at a predetermined position of the grip portion 350.

The cuff main body 340 includes a flexible member 342 for maintaining the tubular form as a core member with the cuff main body 340 being rounded, where the flexible member 342 is covered by an outer cloth 341 and an inner cloth 344. Similar to the cuff main body 40 shown in FIG. 4, a plurality of engagement holes 345 to which the gearing tooth 346 to be described later is arranged along the longitudinal direction of the cuff main body 340 at one end side 340a and the other end side 340b of the cuff main body 340. In the present embodiment, the engagement hole 345 is arranged along two sides in the longitudinal direction of the cuff main body 340.

The cuff main body 340 is rounded to a tubular form so that the upper arm can be inserted from the axial direction, and the grip portion 350 is fixed to the cuff main body 340 so that the grip 353 extends in the direction parallel to the axial direction of the cuff main body 340 formed to a tubular shape. The rotation mechanism 500 is arranged at a position on the outer peripheral surface of the cuff main body 340 and inside the case 351 of the grip portion 350.

The gearing tooth 346 serving as the engagement rotation member is arranged at the position sandwiched by the one end side 340a and the other end side 340b at the position where the one end side 340a and the other end side 340b of the cuff main body 340 overlap each other with cuff main body 340 being rounded to a cylindrical shape. The rotating direction of the gearing tooth 346 is controlled using the rotation mechanism 500.

As shown in FIG. 20, the rotation mechanism 500 includes a geared motor 510, an electromagnetic brake 520, and a rotation shaft 580 including the gearing tooth 346. The geared motor 510, the electromagnetic brake 520, and the rotation shaft 580 are respectively assembled to the supporting frame 546 arranged at a position on the outer peripheral surface of the cuff main body 340 and inside the base 352 of the grip portion 350. The gears 550, 560, 570 serving as a power transmission mechanism are assembled at the predetermined positions of the supporting frame 546.

The geared motor 510 is a motor equipped with a decelerator, and includes a motor portion 510a, a decelerating portion 510b, and an output shaft 510c. A gear 550 is fixed to the output shaft 510c of the geared motor 510. An electromagnetic brake 520 is arranged adjacent to the geared motor 510 at an axial end on the side opposite to the side where the output shaft 510c of the geared motor 510 is positioned. The electromagnetic brake 520 exhibits the braking force with respect to the rotation shaft 510a1 by constraining the rotation shaft 510a1 of the motor portion 510a.

The rotation shaft 580 is fixed to a shaft 557a axially supported by the supporting frame 546, and is driven and rotated when the shaft 557a rotates. The gearing tooth 346 is arranged at both ends of the rotation shaft 580, and an engagement hole 345 of the cuff main body 340 engages with the gearing tooth 346.

A gear 570 is fixed to the shaft 557a to which the rotation shaft 580 is fixed. A gear 560 is fixed to the shaft 560a axially supported by the supporting frame 546. The gear 560 gears with the gear 550 and the gear 570, respectively, and transmits the rotation force generated by the output shaft 510c of the geared motor 510 to the rotation shaft 580. The gears 550, 560, 570 are configured with the respective outer diameter and the number of teeth adjusted, and also function as a decelerator similar to the decelerating portion 510b of the geared motor 510.

The configuration of the function blocks of the blood pressure measurement device 3 according to the present embodiment will now be described with reference to FIG. 21.

The main body 10 includes a CPU 311, an amplifier 320, an A/D (Analog/Digital) conversion circuit 325, a pump drive circuit 321, a valve drive circuit 322, an electromagnetic brake drive circuit 323, and a motor drive circuit 324, in addition to the display unit 14 and the operation unit 16.

The CPU 311 is a means for controlling the entire blood pressure measurement device 3. The memory 326 is configured by a ROM (Read-Only Memory) or a RAM (Random-Access Memory), and is a means for storing programs for causing the CPU 311 etc. to execute the processing procedures for measuring the blood pressure value, and for storing measurement results and the like. The display unit 327 is configured by a LCD (Liquid Crystal Display), and is a means for displaying the measurement results and the like. The operation unit 328 is a means for accepting the operation by the subject and the like, and inputting such command from the outside to the CPU 311.

The CPU 311 inputs the control signal for driving the geared motor 510, the electromagnetic brake 520, the air pump 314, and the air valve 315 to the motor drive circuit 324, the electromagnetic brake drive circuit 323, the pump drive circuit 321, and the valve drive circuit 322, or inputs the blood pressure value serving as the measurement result to the memory 326 and the display unit 327. The CPU 311 also acquires the blood pressure value of the subject based on the pressure value detected by the pressure sensor 313.

The blood pressure value acquired by the CPU 311 is input to the memory 326 and the display unit 327 as the measurement result. The blood pressure measurement device 3 may separately include an output unit for outputting the blood pressure value serving as the measurement result to an external device (e.g., PC (Personal Computer), printer, etc.). A serial communication line, a write device for writing to various types of recording medium, and the like can be used for the output unit.

The motor drive circuit 324 controls the operation of the geared motor 510 based on the control signal input from the CPU 311. The electromagnetic brake drive circuit 323 controls the operation of the electromagnetic brake 520 based on the control signal input from the CPU 311. The pump drive circuit 321 controls the operation of the air pump 314 based on the control signal input from the CPU 311. The valve drive circuit 322 controls the open/close operation of the air valve 315 based on the control signal input from the CPU 311.

The geared motor 510 is an electric motor for rotatably driving the gearing tooth 346 in the forward direction and the reverse direction, and an operation thereof is controlled by the motor drive circuit 324. The electromagnetic brake 520 is a brake that applies a braking force on the gearing tooth 346, and an operation thereof is controlled by the electromagnetic brake drive circuit 323 described above.

The tightening operation of the cuff main body 340 of the blood pressure measurement device 3 according to the present embodiment will now be described. In the blood pressure measurement device 3 according to the present embodiment, the tightening operation with respect to the upper arm 100 of the cuff 300, the measurement operation of the blood pressure value performed after the tightening operation, and the tightening release operation with respect to the upper arm of the cuff 300 performed after the measurement operation are automatically carried out continuously.

The tightening operation with respect to the upper arm 100 of the cuff 300 and the tightening release operation with respect to the upper arm 100 of the cuff 300 are carried out by the tightening operation by the cuff main body 340 by the rotation mechanism 500 and the loosening operation by the rotation mechanism 500 to be described below.

With reference again to FIG. 20, in a state where the geared motor 510 is rotationally driven in the forward direction, the output shaft 510c of the geared motor 510 is rotated in the forward direction, the rotation force is transmitted to the shaft 557a through the gears 550, 560, 570, and the rotation shaft 580 is rotated in the forward direction (direction indicated with arrow in FIG. 19).

When the rotation shaft 580 is rotated in the forward direction, the gearing tooth 346 rotates, and the one end side 340a and the other end side 340b of the cuff main body 340 are sent in the direction where the inner diameter of the tubular form of the cuff main body 340 is reduced through the engagement hole 345 of the cuff main body 340. The tightening operation of the cuff 300 with respect to the upper arm is realized by the sending operation.

At the time of the rotational drive of the geared motor 510 in the forward direction, the electromagnetic brake 520 is in a state where the rotation shaft 510a1 of the motor portion 510a of the geared motor 510 is not restrained, and the motor portion 510a is driven without the operation thereof being limited.

In a state where the geared motor 510 is rotationally driven in the reverse direction, the output shaft 510c of the geared motor 510 is rotated in the reverse direction, the rotation force is transmitted to the shaft 570a through the gears 550, 560, 570, and the rotation shaft 580 is rotated in the reverse direction.

When the rotation shaft 580 is rotated in the reverse direction, the gearing tooth 346 rotates, and the one end side 340a and the other end side 340b of the cuff main body 340 are sent in the direction where the inner diameter of the tubular form of the cuff main body 340 is reduced through the engagement hole 345 of the cuff main body 340. The loosening operation of the cuff 300 with respect to the upper arm is realized by the sending operation.

At the time of the rotational drive of the geared motor 510 in the reverse direction, the electromagnetic brake 520 is in a state where the rotation shaft 510a1 of the motor portion 510a of the geared motor 510 is not restrained, and the motor portion 510a is driven without the operation thereof being limited.

In a state where the geared motor 510 is not rotationally driven in either the forward direction or the reverse direction, that is, when the geared motor 510 is stopped, the rotation shaft 510a1 of the motor portion 510a of the geared motor 510 is restrained by the electromagnetic brake 520.

In the relevant state, the braking force by the electromagnetic brake 520 is applied on the rotation shaft 580 through the rotation shaft 510a1 of the motor portion 510a, the decelerating portion 510b, the output shaft 510c, and the gears 550, 560, 570, and the shaft 570a, so that the rotation operation of the gearing tooth 346 is limited. Therefore, in the relevant state, the tightening operation and the loosening operation of the cuff main body 340 by the gearing tooth 436 are both stopped, and the inner diameter of the tubular form of the cuff main body 340 is maintained constant.

According to the blood pressure measurement device 3 having the above configuration, the rotation shaft 580 is driven in the direction where the one end side 340a and the other end side 340b of the cuff main body 340 further reduce the inner diameter of the tubular form, so that the one end side 340a and the other end side 340b of the cuff main body 340 are simultaneously moved. As a result, the inner diameter of the tubular form of the cuff main body 40 can be easily reduced, and the cuff main body 340 can be easily and rapidly wrapped around the site to be measured. In the control in the blood pressure measurement, the measurement is carried out by the flow similar to that shown in the second embodiment.

Because the air bag 343 is fixed to the base 352 of the grip portion 350, the position of the air bag 343 does not change when wrapping the cuff main body 340 around the upper arm 100, whereby the artery of the upper arm can be accurately compressed, and the accuracy of the blood pressure measurement can be enhanced.

In the embodiment described above, the configuration in which the main body and the cuff are separated is described, but the configuration is not limited to the separated structure, and the present invention can be applied to the blood pressure measurement device in which the main body and the cuff are integrated. When mentioning the number, the amount, and the like in the embodiment described above, the scope of the invention is not necessarily limited to the number, the amount and the like unless particularly stated. It should be recognized that appropriately combining the configuration, the blood pressure measurement control, and the like described in each embodiment above is presumed from the beginning.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1, 2, 3 blood pressure measurement device

10 main body

14, 327 display unit

16, 328 operation unit

20, 300 cuff

30 upper arm supporting stand

31 upper arm supporting surface

31a curved surface

32 case body

33 seat

34 elbow placement section

35 elbow placement surface

35a switch

36 guide roller

40, 340 cuff main body

40a, 340a one end side

40b, 340b other end side

41, 341 outer cloth

42, 342 flexible member

43, 343 air bag

44, 344 inner cloth

45, 345 engagement hole

46, 346 gearing tooth

47, 580 rotation shaft

47a bearing member

48, 64 clutch mechanism

50 handle

60 lock/unlock mechanism

61 slide button

63 engagement shaft

65 engagement pin

66 groove

70 air tube

71 connection cable

100 upper arm

200 torque motor

211, 311 CPU

213, 313 pressure sensor

214, 314 air pump

215, 315 air valve

216 fine tuning lock/unlock mechanism / arm periphery count

320 amplifier

321 pump drive circuit

322 valve drive circuit

323 electromagnetic brake drive circuit

324 motor drive circuit

325 ND conversion circuit

326 memory

350 grip portion

350a first slit

350b second slit

351 case

352 base

353 grip

355 push button

500 rotation mechanism

510 geared motor

510a motor portion

510a1 rotation shaft

510b decelerating portion

510c output shaft

520 electromagnetic brake

546 supporting frame

550, 560, 570 gear

557a, 560a shaft

1. A blood pressure measurement device comprises a cuff with an air bag that compresses an artery of a measurement target site, and that is used by attachment to a site to be measured at the time of measurement, wherein the cuff comprises:
a cuff main body comprising a band-like form in a developed state that is rounded to a tubular form so that one end side and another end side overlap each other to receive the measurement target site from an axial direction; and
an engagement rotation member arranged at a position sandwiched by the one end side and the other end side at a position where the one end side and the other end side of the cuff main body overlap each other with the rounded cuff main body, and arranged to be rotatable in a direction where an inner diameter of the tubular form is reduced or in a direction where the inner diameter is enlarged while engaging with the one end side and the other end side of the cuff main body,
wherein the cuff main body comprises a flexible member for that maintains the tubular form when the cuff main body is rounded.

2. The blood pressure measurement device according to claim 1,
wherein the engagement rotation member comprises a plurality of gearing teeth arranged on an outer surface of a rotation shaft, and
wherein the one end side and the other end side of the cuff main body comprise a plurality of engagement holes with which the plurality of gearing teeth gear along a longitudinal direction of the cuff main body.

3. The blood pressure measurement device according to claim 2,
wherein the plurality of engagement holes is arranged along two sides in the longitudinal direction of the cuff main body in a band-like form, and
wherein the plurality of gearing teeth is arranged at two areas in an axial direction of the rotation shaft so as to gear with the plurality of engagement holes arranged along two sides in the longitudinal direction of the cuff main body.

4. The blood pressure measurement device according to claim 1, further comprising:
an upper arm supporting stand having an upper arm supporting surface for that supports an upper arm that is mounted on a mounting surface,
wherein the cuff main body is arranged at the upper arm supporting stand;
wherein the engagement rotation member is arranged to be positioned on a lower side than the upper arm supporting surface, and
wherein a grip portion is arranged on an outer peripheral surface on an upper side of the cuff main body n a side opposite to the engagement rotation member.

5. The blood pressure measurement device according to claim 4, wherein the upper arm supporting surface is arranged in an inclined manner at an upper part of the upper arm supporting stand so that the upper arm supporting surface is inclined when the upper arm supporting stand is mounted on the mounting surface.

6. The blood pressure measurement device according to claim 1, wherein the rotation shaft includes comprises a lock/unlock mechanism for selecting that selects between a lock state of allowing rotation in a direction where the inner diameter of the tubular form is reduced and inhibiting rotation in a direction where the inner diameter of the tubular form is enlarged, and an unlock state of releasing the lock state.

7. The blood pressure measurement device according to claim 1, wherein the rotation shaft includes comprises a drive device for rotating the rotation shaft.

8. The blood pressure measurement device according to claim 7, wherein the rotation shaft comprises a rotation mechanism that rotates the rotation shaft.

9. The blood pressure measurement device according to claim 1, further comprising:
a housing that holds the cuff main body and accommodating accommodates the engagement rotation member,
wherein the housing comprises:
a first slit that passes one end side of the cuff main body therethrough;
a second slit, positioned on an upper side of the first slit that passes the other end side of the cuff main body therethrough; and
a grip arranged on the upper side of the first slit of the housing.
